(19) United States
(12) Patent Application Publication
(10) Pub. No.: US 2025/0295448 A1
(43) Pub. Date: Sep. 25, 2025

(54) MAPPING AND ABLATION SYSTEM SUITABLE FOR LINEAR PULSED-FIELD CARDIAC ABLATION

(71) Applicant: CRC EP, INC., Lake Oswego, OR (US)

(72) Inventors: Ken NGUYEN, Garden Grove, CA (US); Alan DE LA RAMA, Cerritos, CA (US); Cary HATA, Irvine, CA (US); Tho NGUYEN, Huntington Beach, CA (US); Dorin PANESCU, San Jose, CA (US); Steffen HOLZINGER, Berlin (DE); Sven BODE, Berlin (DE)

(21) Appl. No.: 18/859,962

(22) PCT Filed: Apr. 25, 2023
(86) PCT No.: PCT/US2023/019783

§ 371 Date: Oct. 24, 2024

Related U.S. Application Data
(60) Provisional application No. 63/334,842, filed on Apr. 26, 2022.

(51) Int. Cl. A61B 18/14; A61B 18/00

(52) U.S. Cl. CPC A61B 18/1492; A61B 2018/00077; A61B 2018/00351; A61B 2018/00577; A61B 2018/00613; A61B 2018/00642; A61B 2018/00839; A61B 2018/1467

(57) ABSTRACT

The present disclosure relates to a catheter (C) for ablating a tissue comprising: at least two ablation electrodes (A1, A2) configured for applying a pulse of an electrical energy to the tissue: wherein the catheter is configured such that, in an ablation position of the catheter, the ablation electrodes (A1, A2) contact the tissue along a main axis (z) of the catheter.

### TECHNICAL FIELD

The present invention generally relates to a catheter for ablating a tissue, an according system and a method.

### BACKGROUND

In the medical field, various methods and devices for ablating a tissue are known. Usually, the tissue ablation may be performed for treating and/or preventing various diseases. For example, it is known to ablate cardiac tissue for treating cardiovascular diseases (e.g., cardiac arrythmias, such as atrial fibrillation, ventricular tachycardia, etc.). However, also other types of tissue may be ablated for medical purposes. To enable a reliable ablation treatment, the tissue ablation process usually needs to be controlled in a defined way to ensure a desired medical outcome for the patient. For example, the spatial characteristics of the tissue to be ablated may need to be precisely controlled. To that end, the process evoking the ablation may need to be carefully controlled, e.g. to limit the ablation to a specific tissue type in an active area of the process.

A known approach for tissue ablation is radio frequency ablation (RFA) which is based on applying heat onto the tissue wherein the heat is generated by a current in a radio frequency range. This type of tissue ablation process may be performed by a single-tip catheter with a point-by-point ablation of the tissue (e.g., for cardiac tissue within a heart chamber). The region of ablated tissue may be formed by various ablation points (i.e., ablation sub-areas) that are ablated in consecutive ablation steps of the process such that a total (contiguous) ablation region may be formed. However, RFA treatments are not always optimal. The sequential character of the process may usually require a prolonged processing time. Furthermore, an RFA procedure may lead to a high surgical complexity due to the sequential positioning of the multiple ablation points in the point-by-point application of the process. A reliable positioning of the ablation points may not always be ensured which may for example cause gaps of non-ablated tissue residing within the ablated tissue after the procedure.

Another known approach for tissue ablation is based on a cryogenic process. This may require a cryogenic linear catheter which may apply a cryogenic thermal budget over its tip onto the tissue for its local ablation (e.g., by disposing a cryogenic material). However, due to design constraints, cryogenic linear catheters may be stiff which may cause (surgical) complexities when performing the tissue ablation. Cryogenic linear catheters may also not always be reliably positioned in a target area (e.g., they may slip out of the target area).

Hence, the currently known techniques do not always lead to an optimal ablation of tissue. Therefore, there is a need to find ways to improve the ablation of a tissue.

### SUMMARY

The aspects described herein address the above need at least in part.

A first aspect relates to a catheter for ablating a tissue using pulsed-field (PF) energy configured for connection to a high-voltage generator for generating PF energy, whereby the catheter comprises:
at least two ablation electrodes, in particular at least six ablation electrodes, configured for applying PF energy to the tissue,
wherein the catheter is configured such that, in an ablation position of the catheter, the ablation electrodes contact the tissue along a main axis of the catheter,
wherein the catheter comprises at least two separate electrical conductors to connect to said electrodes to said high-voltage pulse generator for generating PF energy,
wherein the at least two separate electrical conductors are adapted to deliver a waveform with a peak voltage of at least 1000 V, in particular at least 3000 V, to said electrodes, and wherein the ablation electrodes of said catheter may be spaced apart such that the electric field inside cardiac tissue exceeds an intensity of 400 V/cm at a depth of 5 mm when they are energized by said waveform

A second aspect relates a system for ablating a tissue using pulsed-field (PF) energy comprising a catheter and a high-voltage generator for generating PF energy, whereby the catheter comprises:

at least two ablation electrodes, in particular at least six ablation electrodes, configured for applying PF energy to the tissue,
wherein the catheter is configured such that, in an ablation position of the catheter, the ablation electrodes contact the tissue along a main axis (z) of the catheter,
wherein the generator is adapted to connect to said electrodes,
wherein the generator is adapted to deliver a waveform with a peak voltage of at least 1000V, in particular at least 3000 V, to said electrodes, and
wherein the ablation electrodes of said catheter may be spaced apart such that the electric field inside cardiac tissue exceeds an intensity of 400 V/cm at a depth of 5 mm when they are energized by said waveform.

The catheter for ablating a tissue comprises at least two ablation electrodes, in particular at least six electrodes, configured for applying a PF energy to the tissue. Pulsed-field ablation (PFA) renders the targeted tissue non-viable by means of irreversible electroporation (IRE). The electric fields set out by the applied PF energy create pores in the targeted cardiac cell membrane. If the PFA waveform and the characteristics of the catheter are selected appropriately then the pores opened in cell membranes will last long enough to cause cells to program themselves to die. Such process is known as apoptosis. Reference “Modeling Electroporation in a Single Cell. I. Effects of Field Strength and Rest Potential” by DeBruin and Krassowska describes the process in more detail. The catheter may be configured such that, in an ablation position of the catheter, the ablation electrodes contact the tissue along a main axis of the catheter. The distance between the at least two, in particular at least six, electrodes must be selected so that the field created inside tissue exceeds known IRE thresholds (e.g. 400 V/cm). The two ablation electrodes may function as terminals of an electrical circuit wherein a defined voltage and/or a current characteristic (e.g., in form of a pulse) may be set between the two terminals. Hence, by establishing a contact of the ablation electrodes with the tissue, a voltage and/or current pulse between the electrodes can be transferred to the tissue. The electrode configuration of the invention may allow for PF energy to be applied to the tissue that may cause a reaction in the tissue that leads to an ablation thereof, at least in the vicinity of the electrodes and/or in an effective area surrounding the electrodes (e.g. a contiguous area). Adapting a property of the tissue may for example comprise creating local pores in the tissue and/or causing cell death within the tissue.

The ablation electrodes may be positioned along the main axis of the catheter (e.g. at an outer surface of the catheter in an essentially linear fashion, following the main axis, e.g. the longitudinal axis). Hence, the alignment of the electrodes with respect to the tissue can be performed by controlling the main axis of the catheter without a separate controlling mechanism and/or controlling components as would be the case if the electrodes were to be arranged in a more complex position. Hence, the catheter may be provided and used with lower complexity for contacting the ablation electrodes with a tissue in an ablation position. Once the catheter is navigated to the desired location, it may simply be brought in contact with the surrounding tissue, without having to carry out an additional positioning step, as the ablation electrodes may automatically be in an ablation position on the main axis, without first having to fine-position the electrodes, e.g. by expanding further positioning elements. In combination with the applied PF energy provided by the generator the ablation of the tissue along the catheter axis can take place without the need for repositioning of the catheter. The inventive ablation catheter using PFA is intended to render tissues non-viable by irreversible electroporation (IRE). During IRE the electric field provided by the electrodes accommodated along the main axis of the catheter creates pores in cardiac cell membranes. When the number of pores and their sizes are sufficiently great IRE occurs and the cell programs itself to die. Thereby a so-called ablation area is formed along the ablation portion of the catheter (the distal portion of the catheter comprising the ablation electrodes. This may shorten treatment time and ease the requirements as to the skills and training of the physician.

The system and in particular the generator may be configured to deliver high voltage monopolar PF energy or bipolar PF energy or a combination of monopolar and bipolar PF energy as described below. Some examples of applicable waveforms are shown in FIG. 6. Such waveforms, in particular in combination with the arrangement of the electrodes, ensure one-shot application of electrical fields that are high enough to generate therapeutic effects capable of creating moats of conduction block. The generator may comprise an electronic control unit which is adapted to switch between monopolar PF energy and bipolar PF energy supply mode.

The aspects described herein may enable an ablation catheter whose ablation electrodes may be easily and reliably positioned on the tissue by simply positioning the catheter's (sidewall) surface on the tissue, since the ablation electrodes are positioned along the catheter's main axis (in the ablation position). An ablation electrode's contact with the tissue, as well as the ablation region can thus be controlled by (simply) controlling the catheter's main axis. The ablation position may thus be defined as being along the catheter's main axis. In accordance, the ablation region of the tissue may thus also be aligned along the catheter's main axis. The catheter of the present invention may then be held stationary in the according ablation position to perform the application of PF energy to the tissue. Since the ablation region may extend (contiguously) from tissue contacted by a first ablation electrode of the at least two ablation electrodes to tissue contacted by a second ablation electrode of the at least two ablation electrodes, a relatively large region may be ablated in a single shot, without risk of gaps and without having to move or reposition the catheter.

The main axis of the catheter may be curved (e.g., it may not always be aligned along a straight line). To that regard the main axis may be bendable and may be oriented in various ways. The ablation electrodes may be positioned in a portion of the catheter in a vicinity of the catheter tip. In an example, the catheter's main axis in the portion in the vicinity of the catheter tip may be bendable and/or steerable to allow for a precise contacting of the ablation electrodes with the tissue. For example, it may be steerable such that it spans a two-dimensional plane (but it may not allow forming a three-dimensional shape).

An ablation electrode may be formed as a ring electrode which may enclose a circumference of a sidewall of the catheter. An ablation electrode may also be formed as a tip electrode (e.g., covering the tip of the catheter's distal end). Also, an ablation electrode may be formed as a contact electrode which does not (necessarily) enclose the circumference of the catheter (e.g., a sidewall electrode). However, any other type of electrode may be feasible as an ablation electrode. Notably, an ablation electrode should be configured to be capable to sustain the application of PF energy without damaging the ablation electrode. To that regard, an ablation electrode may comprise a stable current conducting material that can reliably sustain the pulse (e.g., gold, platinum, iridium, or a combination thereof). The material may also be adapted such that it does not significantly impair the biological properties of the tissue it is contacting. An ablation electrode may be defined by a certain electrode area that is exposed on an outer surface of the catheter. A part of the electrode area or the complete electrode area may be in contact with the tissue when applying the PF energy.

It may also be conceivable that the ablation electrodes do not necessarily form a direct contact with the tissue to be ablated and may only be positioned in a vicinity of the tissue. However, even in such a scenario, the application of the pulse of the electrical energy may suffice to induce an electrical energy within the tissue to cause an ablation reaction thereof. In such an indirect contact, the gap of the ablation electrode to the tissue may, for example, be bridged by other material (e.g., organic material, e.g., blood).

In an example, the ablation electrodes may directly contact a tissue in the ablation position which is not to be (significantly) ablated. In that example, the tissue may be an intermediary tissue, wherein the pulse is transmitted through the intermediary tissue to tissue that is ablated in the ablation position. For example, the pulse may not exceed an ablation threshold of the intermediary tissue but may exceed or reach an ablation threshold of the (adjacent) tissue such that it may be ablated.

The at least two ablation electrodes that form the terminals for applying PF energy may, for example, comprise two different types of ablation electrodes (e.g., a ring electrode and a tip electrode, a sidewall electrode and a tip electrode and/or electrodes with different electrode areas, etc.).

In an example, the catheter may be configured such that in the ablation position the tissue is ablated along a portion of the main axis, wherein the portion spans at least over a distance between the ablation electrodes. The ablation region of the tissue may thus be shaped based at least in part on the distance between the ablation electrodes. Moreover, the ablation region may be aligned along the main axis of the catheter, since it will be oriented along the vector that is spanned between the at least two ablation electrodes along the main axis. This is because the pulse (e.g., a voltage and/or current pulse) may be applied between the ablation electrodes such that the electrical energy is applied essentially between the ablation electrodes, as well. The ablation reaction may thus be spatially confined to take place at least between ablation electrodes along the main axis. Notably, a specific spatially confined lateral component of the reaction (which may be orthogonal to the main axis) may be present as well, due to the according lateral component of the pulse. This may ensure in total that the tissue is ablated along a portion of the main axis and may be confined to an area where the ablation electrodes reside on the main axis. Since the main axis in the ablation position corresponds to the main axis of the catheter a precise positioning of the ablation regions may be enabled by the present invention.

Notably, the applied pulse of the electrical energy may also be based on the distance between the ablation electrodes that apply the pulse to ensure a desired outcome for the ablation. The ablation reaction of the tissue may depend not only on the pulse characteristics and/or the electrical energy of the pulse but also on the applied electrical field between the ablation electrodes resulting from the pulse. The applied pulse and/or its characteristics may thus be tailored to the distance between the ablation electrodes to ensure a sufficient ablation process of the tissue is taking place.

In an example, the catheter may be configured such that in the ablation position an elongated profile is ablated into the tissue. The elongated profile of the ablated tissue may be defined by a first end and a second end of the elongated shape. The first and second end may be the outermost points of the ablated region on opposite sides. The path between the first and second end may define the length of the elongated shape. A width may be defined in an orthogonal direction to the length, wherein the width may span over a distance of outermost points of the ablation region along the width direction. The elongated shape may be defined that the length of the elongated profile comprises at least two times the width of the elongated profile, preferably at least three times the width of the elongated profile, more preferably at least four times the width of the elongated profile, most preferably at least five times the width of the elongated profile.

In an example of the catheter, at least one of the ablation electrodes may be positioned at a sidewall of the catheter such that it is distanced to a tip of the catheter.

In an example, the ablation electrodes may be arranged in a distal portion of the catheter and distributed over a length of 3 cm to 6 cm, in particular a length of 4 cm to 5 cm. This may lead to an active length of the ablation section between 3 cm and 6 cm, in particular between 4 cm and 5 cm.

In an example of the catheter, the ablation electrodes may be configured for applying an electrical field higher and/or equal to a predetermined threshold to the tissue, wherein the predetermined threshold is associated with an ablation threshold of the tissue. For example, the ablation threshold may comprise a minimum value of an electrical field that is needed to cause an ablation of the tissue. The predetermined threshold of the electrical field to be applied may thus be at least the same as the ablation threshold or higher than the ablation threshold. For example, the predetermined threshold may be chosen to be higher than the ablation threshold to implement a safety margin. This may ensure that the electrical field during the application of the pulse will fulfill the ablation condition (i.e., an electrical field above the ablation threshold) even if (unwanted) variations of the electrical field occur. For example, different safety margins may be chosen for the predetermined threshold (e.g., at least 5%, preferably at least 10%, more preferably at least 15%, most preferably at least 20% of the ablation threshold). The safety margin may be added to the ablation threshold to define the predetermined threshold of the electrical field to be applied to the tissue. The herein described value of the applied electrical field may be the value of the electrical field in an effective ablation area. The effective ablation area may, for example, be an area covered by or in contact with the ablation portion of the catheter. Hence, fulfilling the ablation threshold for the electrical field at least between the ablation electrodes ensures the formation of a desired ablation region defined by the ablation electrodes.

The ablation threshold may comprise a value of 400 V/cm at a depth of 5 mm when they are energized by the waveform provided by the generator. To that regard the electrical field applied to the tissue by the pulse should be higher or equal to 400 V/cm to cause an ablation. With a safety margin of 10% of the ablation threshold the predetermined threshold of the electrical field may be chosen as 440 V/cm. The electrical field to be applied to the tissue should thus, in this example, be higher or equal to 440 V/cm.

In an example, the predetermined threshold may be based at least in part on the tissue to be ablated. For example, the predetermined threshold may be based on the type of tissue (e.g., cardiac tissue, nerve tissue, etc.). Furthermore, the predetermined threshold may be based on the organ that the tissue is surrounding and/or is being a part of (e.g., an atrium, a ventricle, a pulmonary vein, etc.) to ensure the threshold is not set to high.

In an example, the ablation electrodes and the generator may be configured to sustain the applied electrical field that fulfills the ablation threshold condition. Sustaining may comprise that the ablation electrodes may not be significantly damaged by the application of the pulse. The ablation electrodes may also be configured to sustain an electrical field that fulfills a specific safety margin of the ablation threshold condition (e.g., a safety margin of at least 5%, preferably at least 10%, more preferably at least 15%, most preferably at least 20% of the ablation threshold).

In an example, the ablation electrodes and the generator may be configured to (reliably) sustain a predetermined voltage and/or current of the pulse. The ablation electrodes may be configured to sustain the predetermined voltage and/or current over a prolonged period of time, for example, for at least 200 pulse applications, preferably at least 500 pulse applications, more preferably at least 1000 pulse application, most preferably at least 2000 pulse applications to the tissue. The predetermined voltage may comprise a voltage of at least 1000 V, preferably at least 3000 V, more preferably at least 3500 V, more preferably at least 4000 V, most preferably at least 5000 V. In another example, the predetermined voltage may comprise a voltage between 1000 V and 15000 V or between 3000 V and 10000 V, for example. The predetermined current may comprise a current of at least 5 A, preferably at least 10 A, more preferably at least 80 A, most preferably at least 150 A. However, the predetermined current may also comprise a current of at least 200 A. In another example, the predetermined current may comprise a current between 5 A and 200 A, 10 A and 100 A, for example.

The pulse duration of PF energy may comprise a duration of at least 1 μs, at least 5 μs, at least 10 μs, at least 20 μs, or at least 30 μs. In another example, the pulse duration may comprise a duration between 5 μs and 100 μs, for example between 10 μs and 75 μs.

The generator may be configured to applied the PF energy according to the voltage and pulse duration as described above.

The system and in particular the generator may be in particular configured to provide biphasic pulses comprising a positive section comprising the positive pulse peak and a negative section comprising the negative pulse peak. The pulse width is the width of the positive section (or the negative section). Preferably, but not mandatory, the positive and negative phase complex would be charge balanced, so that the net charge delivered to tissue is as close to 0 μC as reasonably possible. Alternatively, the charge-balanced feature may be achieved over the duration of the pulse train. The net charge of the train would, in this case, be as close to 0 μC as reasonably possible. The charge-balanced feature has potential benefits of minimizing bubbling (by lowering chances of electrolysis of the blood), arcing (caused by ionization of the blood or of gases resulted from electrolysis) and skeletal muscle stimulation (direct or indirect via motor nerves). A biphasic pulse starting with a positive or negative section is understood as positive or negative (biphasic) pulse.

According to an embodiment, positive and negative pulses are separated by the interphase delay. The advantage of the pulse width according to the invention is that the electric field acts sufficiently long against the cells so that pores are created by the electric field. The interphase delay may be chosen in the region of 100 ns to 100 μs, in particular in the region of 500 ns to 50 μs, so that the negative phase does not cancel too soon the effects of the positive phase and that the interphase delay is not too long. If the interphase delay becomes too long, the charge balance does not work. Negative and positive phases may be provided with the same amplitude or with a different amplitude, as long as a charge-balanced pulse train are achieved.

According to an embodiment, consecutive biphasic pulses are delivered, The interpulse delay between two consecutive biphasic pulses may be in the region of 100 μs to 3 ms, in particular in the region of 500 μs and 2.5 ms, in particular in the region of 1.5 ms to 2.5 ms. A sequence of consecutive biphasic pulses could be considered as a pulse train. Such a pulse train may comprise 5 to 20, in particular 8 to 12, biphasic pulses. The generator may be configured to receive a medical signal, in particular a signal indicating the heat beat, and synchronize the application of the pulse trains to medical signal, in particular to the beat of the heart. One pulse train as disclosed above may be applied with each beat of the heart for 50 to 200 heart beats, in particular for 100 to 150 heart beats. After a pause of several seconds up to several minutes the above disclosed sequence may be repeated. The peak amplitude of the biphasic pulses may be in the region of 3 kV to 5 kV, in particular in the region between 3.5 kV and 4.5 kV, in particular between 3.8 kV and 4.2 kV.

In an embodiment using biphasic pulses the interphase delay is determined between two consecutive biphasic pulses, where a biphasic pulse is followed by an inverse biphasic pulse (for example a negative biphasic pulse following a positive biphasic pulse). The time between the first biphasic pulse and the start of the following inverse biphasic pulse is the interphase delay and as well within the range of 1 μs to 100 μs.

In an example, the catheter may comprise two or more pairs of ablation electrodes positioned on or along the main axis.

At least two of the pairs may be configured (or configurable, e.g. by a corresponding switch or other suitable configuration element: in the following the term configured will be used but the term configurable is implied as well even if this is not expressly stated) for applying separate pulses of the electrical energy to the tissue in the ablation position. The catheter may thus not be limited to applying a single pulse via the terminals of two ablation electrodes. According to the invention, the catheter may also comprise multiple ablation electrode pairs wherein each pair comprises two ablation electrodes such that each pair may apply a separate pulse to the tissue.

In an example, each pair may be separately controlled such that each pair may apply a separate pulse independent from the pulse output of other pairs of the catheter. For example, the catheter may comprise three electrode pairs. In that example, the catheter may be configured to independently apply a pulse from the first pair, the second pair and/or the third pair. For example, the catheter may be configured to enable exclusively applying a pulse from the second pair without applying a pulse from the first and third pair. In that example, the catheter may also be configured to enable independent pulse characteristics of the applied pulses from the electrode pairs. For example, the second pair may apply a pulse with a maximum voltage of 3500 V, wherein the first and third pair may apply a pulse with a maximum voltage of 3000 V. The separate control of the pairs may be accomplished by an according circuitry in the catheter such that each pair may be separately addressed for providing the respective separate pulse.

In an example, the catheter may be configured such that at least two of the pairs (of ablation electrodes) apply a pulse of an electrical energy substantially simultaneously to the tissue. This may enable to cover a larger ablation region with one-shot (i.e., one simultaneous application of pulses) compared to only using one pair of electrodes. In that example, the ablation region may thus be defined by the at least two pairs of ablation electrodes that are aligned along the catheter's main axis. For example, the ablation region may be formed at least along the connecting paths of the ablation electrodes of each of the pairs of the at least two pairs. In an example, the at least two pairs may be adjacent pairs of ablation electrodes on the catheter's main axis. This may, for example, ensure the formation of an ablation region spanning at least from a first ablation electrode of the first pair to a second ablation electrode of the second pair along the main axis.

In an example, the catheter may be configured such that at least three, at least four, at least five, or at least six of the pairs (of ablation electrodes) apply a pulse of an electrical energy substantially simultaneously to the tissue.

In an example, the two or more pairs of ablation electrodes may have one or more ablation electrode in common. To illustrate an example, two pairs of ablation electrodes may be formed by three ablation electrodes (e.g., a first, a second and a third ablation electrode). For, example, the second ablation electrode may be shared among the pairs. To that regard, the first pair of ablation electrodes may comprise the first and second ablation electrode, wherein the second pair of ablation electrodes may comprise the second and third ablation electrode.

In an example, the two or more pairs of ablation electrodes may be positioned along the catheter's main axis, such that the ablation electrodes can be positioned along a line (e.g., a straight line and/or a curved line) onto the tissue. The catheter may thus cause a linear shape (e.g., an elongated shape as described herein) of an ablation region since the ablation region may be defined by the contact positions of the ablation electrodes of the two or more pairs on the tissue. The ablation region may, for example, correspond to a lesion for therapeutical purposes. Hence, linear shape lesions may be formed by using the catheter according to the invention. Moreover, straight lines and/or curved lines of ablation regions (e.g., lesions) may be formed in the tissue with a high flexibility and precision. The linear shape of the ablation region may be formed by shaping the orientation of the portion of the main axis of the catheter that comprises the two or more pairs of ablation electrodes. A curved linear shape of the main axis may result in an assembly of ablation electrodes contacting the tissue in a corresponding curved linear order. Hence, by applying pulses for ablation via the two or more pairs a curved linear shape may be formed as an ablation region. A (substantially) straight linear shape of the main axis may result in an assembly of ablation electrodes contacting the tissue in a corresponding (substantially) straight linear order. Hence, by applying pulses for ablation via the two or more pairs a (substantially) linear shape may be formed as an ablation region.

Notably, the line and/or linear shape may be formed without moving the catheter during the ablation process which may reduce the surgical complexities significantly. Moreover, a repositioning of the main axis during an ablation procedure to form the ablation region may also not be necessary compared to known approaches. The shape of the ablation region may be set by the orientation of the catheter's main axis.

The ablation region may be evoked as long as the contact of the ablation electrodes to the tissue is ensured before the application of the pulses. The linear shape of the ablation region may, for example, be evoked by a substantially simultaneous application of the pulses via the ablation electrode pairs. However, since the catheter may be stationarily fixed in the ablation position, also a sequential application of the pulses by the two or more pairs may result in a linear ablation region.

In an example, the two or more pairs of ablation electrodes may be positioned along the main axis such that the application of the electrical energy in the ablation position causes a contiguous elongated profile without gaps ablated in the tissue. The ablation electrodes may be distanced with respect to each other, such that the application of pulses via the two or more pairs does not leave areas (e.g., gaps) within the ablation region that did not receive the ablation threshold (as described herein). A gap may be understood as a non-ablated region within the ablated region. A gap may also be understood as a partial fracture formed by a non-ablated region passing through the ablation region such that at least two separate ablated regions are formed (e.g., if the fracture of non-ablated tissue spans along the entire width of the ablated region). Ensuring that no gap formation is present may be highly advantageous since it may be necessary for medical purposes to form a contiguous elongated (line shape) profile that may function as an electrical isolation. For example, the ablation region may be formed such that an electrical signal within the tissue cannot pass through the ablation region. By ensuring that no gap formation is present in the ablation region said function of electrical isolation may be reliably enabled. The gap formation may be an interplay of the distance between ablation electrodes, the ablation threshold and the applied pulse. Suppressing the gap formation may thus be enabled by adapting the electrical characteristics of the pulse applied between two ablation electrodes based on the distance between the ablation electrodes, or vice versa. For example, a higher distance between ablation electrodes may require higher voltages and/or currents in the pulse to induce a high enough electrical field such that the ablation threshold condition is fulfilled in the tissue. For example, a lower distance between ablation electrodes may require lower voltages and/or currents in the pulse to induce a high enough electrical field such that the ablation threshold condition is fulfilled in the tissue.

In an example, the catheter may comprise one or more mapping electrodes for sensing of the tissue, in particular to sense intracardiac electrograms. A mapping electrode may be configured for sensing an electrical activity of the tissue. The one or more mapping electrodes may be positioned along the main axis of the catheter. For example, the mapping electrodes may be positioned in the portion of the main axis of the catheter that comprises the ablation electrodes. Hence, the mapping electrodes may enable to sense the tissue in the active area that is to be ablated or was ablated by the ablation electrodes. The mapping electrodes may thus be used to assess the tissue prior to applying the pulse (e.g., for determining the medical situation prior to the surgery). On the other hand, the mapping electrodes may be used to assess the tissue after the pulse and/or after the pulses have been applied, for example to determine if a successful ablation of the tissue has occurred. This may be highly advantageous for medical purposes since the mapping electrodes constitute an in-situ detection mechanism with respect to the ablation procedure. Hence, the invention may enable to omit the need for a separate detection device or procedure to assess the ablation. Also, the mapping electrodes may deliver sensing signals before and/or after the ablation without having to move the catheter out of the ablation position.

In an example, two mapping electrodes on the main axis (e.g. as described with respect to the ablation electrodes) may be configured to function as a bipolar sensor. In another example, a mapping electrode on the main axis may be configured to function as a unipolar sensor wherein the other electrode of the unipolar sensor may reside in another part of the catheter, or a component connected to the catheter.

In an example, the catheter may comprise at least one pair of mapping electrodes wherein the pair comprises two mapping electrodes. In another example, the catheter may comprise at least two pairs of mapping electrodes, preferably at least three pairs of mapping electrodes, or at least four pairs of mapping electrodes, or at least five pairs of mapping electrodes.

As described herein, the ablation may, for example, be performed for a (local) electrical isolation of the tissue. In such a case, a successful ablation may be verified by a signal of the one or more mapping electrodes that signifies that no (significant) electrical activity is taking place in the ablation region anymore. Such a verification may, for example, be performed by comparing the signal of the one or more mapping electrodes prior to the ablation with the signal of the one or more mapping electrodes after an ablation procedure.

The mapping electrodes may be configured for detection of a desired (electrical) signal that is passing through the tissue which can be used to assess the ablation procedure. However, the mapping electrodes may also be configured to suppress an undesired signal which may be associated with a particular organ that may crosstalk its (electrical) signal to the tissue in the ablation region which, however, is not associated with the ablation procedure. For example, the electrode area of the mapping electrode may be chosen to be limited in size (e.g., compared to the electrode area of an ablation electrode) to suppress the sensing of parasitic signals and enhance the local sensing of the tissue in the ablation region. For example, in a medical application of the catheter, the ablated tissue may comprise a cardiac tissue (e.g., a tissue from an atrium, a ventricle, etc.). The mapping electrodes may be made sufficiently small such that the electrical activity of heart components (e.g., the atrium, the ventricle) that are not associated with the ablation procedure of the cardiac tissue may not significantly crosstalk to the mapping electrodes.

For example, mapping electrodes may be positioned along the main axis of the catheter distally from the ablation electrodes and/or proximally from the ablation electrodes and/or in between neighboring pairs of ablation electrodes.

In some examples, at least one (or all) of the one or more mapping electrodes may have a surface area that is smaller than at least one (at least two or all) of the at least two ablation electrodes. A mapping electrode may have a length of less than 3 mm along the catheter axis, in particular between 0.5 mm and 2.5 mm, in particular between 0.5 mm and 2 mm, in particular between 1.0 mm and 1.5 mm. An ablation electrode may have a length of at least 3 mm along the catheter axis.

The sensory evaluation of the mapping electrodes may be performed by a computing entity connected to the catheter that may receive the signals of the mapping electrodes (e.g., comprised by a power unit of the system according to the second aspect as described herein).

In an example of the first aspect of the invention, the system and/or the catheter may be configured for switching a mapping electrode to function as an ablation electrode; and/or switching an ablation electrode to function as a mapping electrode. To that regard an electrode of the catheter may be used once as a mapping electrode and once as an ablation electrode. In an example, the catheter may comprise at least a first pair and a second pair of mapping electrodes that may be configured to form at least two bipolar sensors on the main axis. However, the at least first and second pair of mapping electrodes may also be reconfigured to function as two ablation electrodes (i.e., as one pair of ablation electrodes). To that regard, the two electrodes of the first pair of mapping electrodes may be reconfigured as a combined ablation electrode. For example, the two electrodes of the first pair may be set on the same polarity such that the first pair forms the lower polarity ablation electrode of an ablation electrode pair (e.g., with the lower polarity −). The two electrodes of the second pair of mapping electrodes may then be reconfigured to function as the other ablation electrode of said ablation electrode pair. For example, the electrodes of the second pair may be set to the same polarity, such that they may form the higher polarity ablation electrode (e.g., the higher polarity +). In an example where at least two adjacent mapping electrodes are arranged as pair of mapping electrodes. Two adjacent mapping electrodes may be separated by an inter-mapping spacing of less than 3 mm to function as an ablation electrode. In particular two adjacent mapping electrodes are separated by an inter-mapping spacing which is between 0.2 mm and 2.5 mm, in particular between 0.4 mm and 2.0 mm, in particular between 0.5 mm and 1.0 mm.

A pair of ablation electrodes may be arranged on the catheter in a way that the two adjacent ablation electrodes of the pair are separated by an inter-pair distance of at least 3 mm. In particular, the two ablation electrodes of a pair of ablation electrodes may be separated by an inter-pair distance between 3 mm and 10 mm, in particular between 3 mm and 6 mm, in particular between 3 mm and 4 mm. Each ablation electrode of a pair of ablation electrodes may be either an ablation electrode or two adjacent mapping electrodes arranged as pair of mapping electrodes and functioning as ablation electrode.

In an embodiment six pairs of mapping electrodes may be arranged on the catheter to function as three pairs of ablation electrodes.

This may enable using the same electrodes for different purposes, while still tailoring the electrode configuration for each purpose. For enabling a sufficient tissue ablation, the area of the ablation electrodes usually needs to be sufficiently large to sustain the electrical energy of the ablation pulse and to apply the electrical energy efficiently to the tissue. However, for a sensing application the area of a mapping electrode should be in comparison smaller, for example, to avoid the effects of parasitic crosstalk signals. By combining two mapping electrodes to a single ablation electrode, an ablation electrode with a wider area can be formed to enable the herein described boundary conditions for an improved ablation electrode. However, by switching back to the mapping electrode configuration, the smaller electrode areas for sensing purposes may be enabled. Hence, improved ablation and mapping/sensing capabilities may be achieved even with (substantially) the same electrodes (and/or electrode areas) positioned on the main axis.

The switching between a mapping and an ablating configuration may be performed by a computing entity that may be connected to the catheter (e.g., comprised by a power unit of the system according to the second aspect as described herein that may power the catheter and particularly supply the voltage/current for applying the pulses).

Notably, the features relating to switching between mapping and ablating electrodes may be used independently from other features of the first aspect (e.g. irrespective of whether the ablation electrodes contact the tissue along the main axis in the ablation position).

In an example, at least one mapping electrode may be positioned between two ablation electrodes. This may allow for an improved sensing as the signal may be picked up within the ablation region, since the path between two ablation electrodes may be ablated as described herein. In an example, at least one mapping electrode pair may be positioned between two ablation electrodes. It may also be conceivable that at least one mapping electrode (and/or at least one mapping electrode pair) is positioned between each pair of ablation electrodes or each second pair of ablation electrodes.

In an example, a mapping electrode pair may be positioned at a distal end of the catheter (e.g., such that no further ablation electrode is positioned between the mapping electrode pair and the tip of the catheter). In another example, a mapping electrode pair may be positioned proximally (e.g., such that the ablation electrodes are positioned between the mapping electrode pair and the tip of the catheter).

In an example, the catheter may be configured to allow for bridging of the pulse over at least one mapping electrode that may be positioned between two ablation electrodes. For example, the pulse applied between two ablation electrodes that have a mapping electrode between them may comprise a higher voltage and/or a higher current than a pulse applied between ablation electrodes that have the same distance between them, but no mapping electrode positioned in between (e.g., as controlled by a power unit of the system according to the second aspect as described herein).

In an example, the catheter may comprise at least one pair of mapping electrodes and one pair of ablation electrodes, wherein the electrical conductors connected to the mapping electrodes are in a separate lumen of the catheter than the electrical conductors connected to the ablation electrodes and wherein conductors connected to mapping and/or ablation electrodes of opposite polarity do not share a lumen The electrical conductors connecting the mapping electrodes electrically to the generator or a mapping system are referred to as mapping wires as well. The electrical conductors connecting the ablation electrodes electrically to the generator are referred to as ablation wires as well. Mapping wires of different (opposite) polarities may be in separate lumens, whereby mapping wires of the same polarity can share a lumen. The same may be even more important for ablation wires of opposite polarity. Ablation wires and/or mapping wires may be coated with an insulating material as well.

The individual lumens may be arranged to maximize the distance between opposite polarities to withstand voltages in excess of 10 kV. Thereby all mapping wires sharing the same polarity may be arranged in one lumen separate of the lumen for the mapping wires of the opposite polarity. The ablation wires sharing the same polarity may be arranged in one lumen separate of the lumen for the ablation wires of the opposite polarity as well. This may lead to four separate lumens for the electrical conductors, whereby the two lumens for one group of mapping wires having the same polarity as one group of ablation wires may be arranged as far away from the two lumens for the group of mapping wires and ablation wires of the opposite polarity.

Such an arrangement ensures a safe distance between the mapping wires and the ablation wires to avoid any pick-up of high-voltage from ablation wires by the mapping wires. Pick-up high voltages could cause field distortion, ineffective delivery of high voltage and/or shunting during the ablation procedure.

In an alternative embodiment, mapping wires and ablation wires of the same polarity may be arranged in one lumen separate of the lumen for the mapping and ablation wires of the opposite polarity. Such an arrangement might be advantageous in a system using voltages in the lower kV range, where a small catheter profile is desired.

In an example of the invention, the catheter may be configured for steering of an orientation of the main axis of the catheter. For example, the catheter may comprise a steering element that may allow to curve the main axis of the catheter. The steering may be performed especially in the portion that comprises the ablation electrodes and/or the mapping electrodes. The steering of the orientation may be performed two-dimensionally. For example, in a basic undeflected configuration the catheter's main axis may coincide with a longitudinal (straight) axis of the catheter. The steering may enable to curve the main axis with respect to the initial (undeflected) longitudinal axis. The steering may enable to form a curve of the main axis in a sectioned path that may be described up to a quarter circle section, up to a half circle section, up to a three-quarter circle section and/or up to a four-fifth circle section. The catheter may not be steerable such that a closed loop is formed and/or such that the steerable portion forms a three-dimensional structure (e.g. extending out of a plane). The main axis may thus be steered to curl in a certain direction. The tangent of the initial point of the catheter (at which the curl essentially starts) and a line connecting the initial point of the catheter and a tip of the catheter may span a curl angle that may define the curl. For example, the curl angle may be up to 5°, up to 10°, up to 45°, up to 90°, up to 135°, up to 180°. In an embodiment a pull wire is used as steering element to steer the ablation portion of the catheter as described above.

In another example, the steering may be performed especially in a portion proximal the portion that comprises the ablation electrodes and/or the mapping electrodes. The steering may enable to form a curve of the main axis in a sectioned path that may be described up to a quarter circle section, up to a half circle section, whereby the curve is located proximal and adjacent to the ablation section (the section of the catheter from the most proximal electrode functioning as ablation electrode to the most distal electrode functioning as ablation electrode). In this example the ablation section would be a straight (linear) portion of the catheter and the adjacent proximal section would be steerable to form a curve. In this example the contact between the ablation section and the tissue could be improved.

In an example stiffness of the distal section of the catheter comprising the ablation section may be increased with respect to the proximal section of the catheter, which may further improve the contact between the ablation section and the tissue. The stiffness of the distal section comprising the ablation section may correspond a shore hardness of at least 55D, in particular a shore hardness of at least 72D. The distal section may comprise a nitinol wire to increase the stiffness of the distal section with respect to the proximal section. The nitinol wire may have a diameter in the region of 0.2 mm to 0.8 mm, in particular in the region of 0.55 mm to 0.65 mm. In addition or as an alternative, at least one of the lumens comprising the ablation wire(s) and/or mapping wire(s) may comprise an additional polymer tube in the distal section to increase the stiffness.

In an example of the first aspect, the catheter may be configured for applying a pulse that causes irreversible electroporation of the tissue. For example, pulsed-field application (PFA) treatment may be based on irreversible electroporation (IRE). The herein described catheter may thus be used for applying PFA treatment to the tissue.

In another example, the catheter may be configured such that a thermal ablation may (substantially) not be induced by the applied pulses in the tissue. For example, radio frequency ablation (RFA) treatment may rely on inducing heat into the tissue for ablation thereof. However, the catheter according to the invention may be configured to apply pulses for non-thermal ablation of the tissue since thermal ablation of the tissue may not always be medically optimal and/or useful.

In an example, the catheter may comprise a connector for connecting the electrodes of the catheter to the generator to deliver the PF energy to the electrodes. The connector may enable the control lines of the catheter to be safely connected to the generator. For example, the connector may enable a reliable connection of the ablation electrodes and/or the mapping electrodes to the power unit. Furthermore, the connector may enable a reliable connection of the steering mechanism (e.g., steering element) to the power unit to control the steering of the catheter's orientation. In an alternative embodiment the connector may provide a connection of the electrodes to a specific mapping system, in particular a mapping system configured to display intracardiac electrograms.

An alternative aspect of the invention relates to a catheter for sensing a tissue comprising: at least two mapping electrodes configured for sensing an (electrical) activity of the tissue, wherein the mapping electrodes are positioned along a main axis of the catheter, wherein the catheter is configured such that, in a sensing position of the catheter, the mapping electrodes contact the tissue along the main axis. The catheter according to the invention may thus also be used as a sensor device and may not be limited for the use of an ablation catheter. In an example, of the alternative aspect the ablation electrodes may be omitted. However, all aspects described for the catheter of the first aspect may be accordingly applied for the sensing catheter of the alternative aspect.

The system and in particular the generator may be configured to apply PF energy to the tissue. For example, the generator may comprise a power electronic circuitry to enable setting the pulse characteristics of the pulses applied via the one or more ablation electrodes of the catheter. The generator may also be connected to a computer (e.g., a microprocessor, an ASIC, a computing device, etc.) for controlling the catheter and/or the power electronic circuitry. For example, the computer may be used to set a certain pulse characteristic that may be processed by the power electronic circuitry such that an according pulse is applied via the ablation electrodes. The computer may also be configured to apply control instructions to the steering mechanism of the catheter (e.g., to curl the catheter in a desired curl angle). Furthermore, the computing entity may be configured to receive data of the catheter (e.g., sensing data of the mapping electrodes). The computer may thus also be used for signal processing of the signals sensed by the mapping electrodes (e.g., via signal processing algorithms). The computer and/or the generator may comprise a user interface (e.g., a monitor, a keyboard, a touchscreen, etc.) such that a user may interact with the system.

The computer may comprise one or more storage devices that may store one or more instructions that may be executed by the computer to perform the herein described functions of the system and/or catheter via corresponding method steps.

A third aspect relates to a method for ablating a tissue comprising: positioning an ablation catheter in an ablation position such that at least two ablation electrodes positioned on or along a main axis of the catheter contact the tissue along the main axis: applying a pulse of electrical energy via the at least two ablation electrodes to the tissue. The catheter of the method may comprise a catheter according to the first aspect. The method may be performed by the system according to the second aspect.

A fourth aspect relates to a computer program comprising instructions that, when executed by a computer, a system according to the second aspect and/or a computing device of the system, cause the computer, the system and/or the computing device to perform the method and/or a method step according to the method of the third aspect. In an example, the computer program may comprise instructions that may cause the performing of various other tasks, control functions and/or data analysis of the catheter and/or the system as described herein.

It is noted that the method steps as described herein may include all aspects described herein, even if not expressly described as method steps but rather with reference to a catheter (or system or device or computer). Moreover, the catheters, systems and computer programs as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

### DESCRIPTION OF THE DRAWINGS

FIG. 1: Schematic representation of exemplary embodiments of the catheter according to the present invention.

FIG. 2: Simulation results for the exemplary embodiments of the catheter representing the interaction of the exemplary embodiments with the tissue for various pulse parameters.

FIG. 3: An exemplary embodiment of a catheter according to the invention with a curved main axis.

FIG. 4: Representation of an exemplary catheter ablation position in a heart model for an exemplary ablation treatment of the heart.

FIG. 5: Representation of an exemplary pulse which may be applied by a pair of ablation electrodes of the catheter for tissue ablation.

FIG. 6: Representation of another exemplary pulse which may be applied by a pair of ablation electrodes of the catheter for tissue ablation.

FIG. 7: Representation of 3D voltage maps showing a conductivity before and after a CTI ablation procedure in the right atrium, as well as a pathological overview of the ablation area.

FIG. 8: Schematic representation of an exemplary embodiment of the system according to the invention

FIG. 9a, b, c: Schematic representation of exemplary embodiments of the lumen arrangement of a catheter according to the present invention

### DETAILED DESCRIPTION

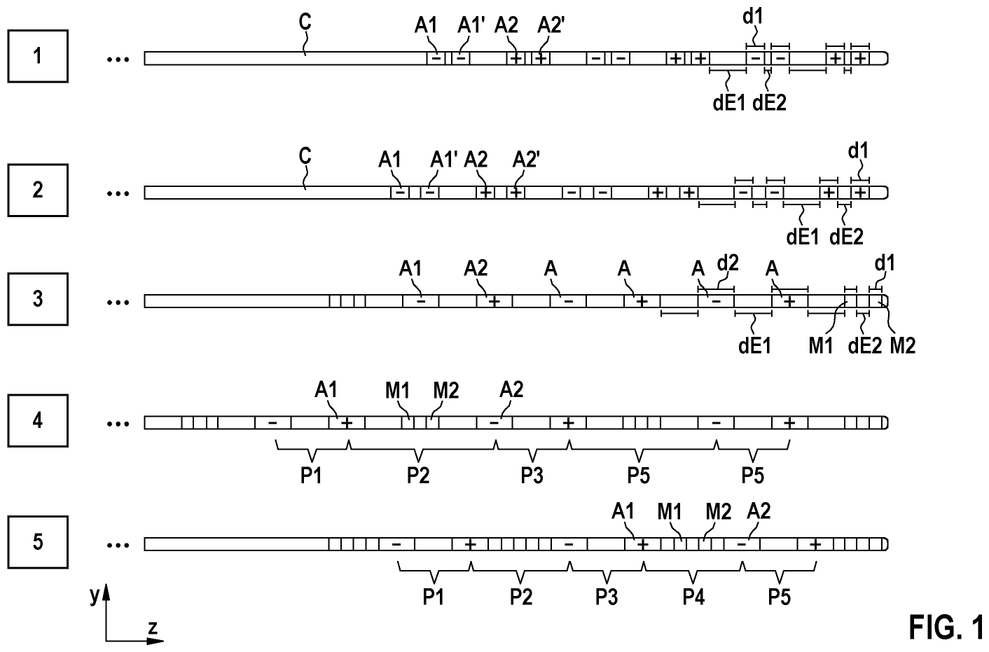

FIG. 1 shows a schematic representation of five exemplary embodiments 1, 2, 3, 4, 5 of a catheter C. The exemplary embodiments may also be referred herein as catheter 1, catheter 2, catheter 3, catheter 4, catheter 5 according to the embodiment's number. In FIG. 1, a portion of the catheter's main axis z at the distal end is shown that may comprise ablation electrodes, as well as mapping electrodes according to the invention. The ablation electrodes and mapping electrodes may be positioned in various ways on or along the catheter's main axis and the possible positions are not limited to the shown exemplary embodiments.

To facilitate a general understanding, exemplary embodiment 3 of catheter C is discussed first. As can be seen in FIG. 1, catheter 3 may comprise six ablation electrodes. In other examples, more or less than six ablation electrodes may be provided. Each two adjacent ablation electrodes may form a pair of ablation electrodes that may apply a pulse of an electrical energy to a tissue for ablation purposes, as described herein. An exemplary pair of ablation electrodes A1, A2 is accordingly labelled on catheter 3, wherein the remaining ablation electrodes are simply labelled as A. However, the characteristics of the ablation electrodes A1, A2 may also accordingly apply to the further ablation electrodes A, even if not expressly stated in regard to each single aspect hereinbelow.

The ablation electrode A1 may be connected to a lower polarity −, wherein the ablation electrode A2 may be connected to a higher polarity +. For example, the lower polarity − may be considered a reference potential (e.g., ground), wherein the higher polarity + may be considered the terminal that applies an electrical signal with respect to the reference potential. Hence, a defined electrical pulse may be applied between the ablation electrodes of a pair of ablation electrodes A1, A2 via the catheter C. The same may apply to the two pairs formed by the four further ablation electrodes A.

Moreover, the defined electrical pulse may be shaped to enable a pulsed-field ablation (PFA) treatment of the tissue. The ablation electrodes may thus be configured to apply a PFA pulse to apply PFA energy to the tissue. In a preferred example, the ablated tissue may comprise a cardiac tissue. In that case the ablation may comprise causing irreversible electroporation of the cardiac tissue via the PFA treatment. The ablated tissue may in that case be regarded as a lesion.

The ablation electrodes A1, A2, A of the catheter may be positioned in a string of alternating polarities along the main axis, as shown for catheter 3. Hence, between every pair of consecutive ablation electrodes (A1, A2), (A2, A1), . . . , an electrical pulse can be applied to the surrounding tissue along the main axis z of the catheter C. In an example, catheter C may apply the pulses via the ablation electrodes substantially simultaneously. The catheter C may thus constitute a one-shot linear ablation catheter. For example, the pulse applied by the ablation electrodes A1, A2, A may be shaped such that one (simultaneous) application of the pulses may suffice to ablate the surrounding tissue. The tissue may thus be ablated in a linear (elongated) shape, as described herein. In other examples, also a sequence of simultaneous pulses (e.g. a pulse train) may be applied.

The ablation electrodes A1, A2, A may comprise a specific ablation electrode length d2 with respect to the catheter's main axis z. The ablation electrodes A1, A2, A may for example comprise ring electrodes wherein the specific ablation electrode length d2 may scale the area of the ring electrode. The distance between two ablation electrodes A1, A2, A on the main axis z may be referred to herein as an interpair spacing dE1 (understood as distance from one end of an ablation electrode to the closest end of a neighbor ablation electrode). The interpair spacing dE1 may be an important parameter for shaping the pulse to be applied as the interpair spacing dE1 may impact the applied electrical field strength to the tissue based on a given voltage difference between a pair of ablation electrodes.

In an example, the interpair spacing dE1 may be at least smaller than 10 mm, preferably at least smaller than 5 mm, more preferably at least smaller than 4 mm, most preferably at least smaller than 3.5 mm, for example about 3 mm. This may ensure effective PFA therapeutic effects.

In an example, the specific ablation electrode length d2 may be at least smaller than 20 mm, at least smaller than 10 mm, at least smaller than 4 mm, at least smaller than 3.5 mm or for example about 3 mm. Ablation electrodes of catheter C may comprise the same or different lengths.

Catheter C may further comprise mapping electrodes. To that regard, catheter 3 may for example comprise mapping electrodes M1, M2 positioned at the distal end and/or mapping electrodes M1, M2 at the proximal end of the catheter C. The mapping electrodes M1, M2 of the catheter at each end may be configured as a bipolar sensor for sensing the surrounding tissue. The mapping electrodes M1, M2 may be disabled during the application of the ablation pulses via the ablation electrodes. However, the mapping electrodes may also be active for sensing during the application of the pulses. This may, for example, be implemented for catheter 3 since the mapping electrodes are not positioned between ablation electrodes such that a sensing may be feasible during the application of an ablation pulse.

The mapping electrodes M1, M2 may comprise a specific mapping electrode length d1 with respect to the catheter's main axis z. The mapping electrodes M1, M2 may comprise ring electrodes wherein the specific mapping electrode length d1 may scale the area of the ring electrode. The distance between two mapping electrodes M1, M2 on the main axis z may be referred to herein as an inter-mapping spacing dE2.

In an example, the inter-mapping spacing dE2 may be at least smaller than 3 mm, at least smaller than 2 mm, at least smaller than 2 mm, or smaller than 0.5 mm. In catheter 3, the inter-mapping spacing dE2 may comprise about 1 mm.

In an example, the specific mapping electrode length d1 may be at least smaller than 3 mm, at least smaller than 2 mm, at least smaller than 1 mm, or at least smaller than 0.5 mm. This may ensure a high-resolution mapping. In catheter 3, the specific mapping electrode length d1 may comprise about 1 mm.

A catheter C according to the invention may thus comprise means for ablation (e.g., ablation electrodes A1, A2, A), as well as means for sensing the surrounding tissue (e.g., mapping electrodes M1, M2).

The catheters C according to the five embodiments 1, 2, 3, 4, 5 are particular suitable for providing biphasic PF energy as described above. The catheters according to the embodiments 1, 2, 3 are suitable for providing monopolar PF energy as well.

Subsequently, the exemplary embodiment of catheter 1 will be discussed. Catheter 1 may constitute an example wherein the catheter comprises mapping electrodes that may be switched to function as ablation electrodes and vice versa. To that regard catheter 1 may comprise several pairs of mapping electrodes, wherein FIG. 1 exemplarily depicts six mapping electrode pairs. A first pair of mapping electrodes A1, A1′ and a second pair of mapping electrodes A2, A2′ are labelled in FIG. 1. The inter-mapping spacing dE2 of catheter 1 may, for example, be about 0.5 mm (preferably <1 mm) to ensure high-resolution mapping. The specific mapping electrode length d1 of catheter 1 may comprise about 1.5 mm (preferably <2 mm). When functioning as mapping electrodes, the mapping electrode A1 may represent the lower polarity −, wherein the mapping electrode A1′ may represent the higher polarity + of the bipolar sensor formed by the first pair. Also, in that case, the mapping electrode A2 may represent the lower polarity −, wherein the mapping electrode A2′ may represent the higher polarity + of the bipolar sensor formed by the second pair. This assembly may be beneficial since a high-resolution mapping along the main axis z may be performed via the mapping electrode configuration.

However, the polarities depicted in catheter 1 represent the polarities of the electrodes of the catheter C when they are switched such that they may function as ablation electrodes. This may be performed by the system (as described herein) that may connect the electrodes to specific polarities wherein the polarities may be driven/controlled by the system's power unit (e.g., which may comprise a PFA generator for applying pulse-field ablation pulses). In that case, the first pair of mapping electrodes A1, A1′ may function as a first ablation electrode of an ablation electrode pair. The second pair of mapping electrodes A2, A2′ may function as a second ablation electrode. To enable such a function, the electrodes A1, A1′ may both be switched to the lower polarity − (e.g., representing the reference potential), wherein the electrodes A2, A2′ may both be switched to the higher polarity + (e.g., the signal potential of the pulse). Hence, an ablation electrode pair may be formed with an interpair distance/spacing of dE1. The interpair spacing dE1 of catheter 1 may, for example, comprise about 3 mm (also other values such as those described with reference to embodiment 3 of FIG. 1 may be possible).

Therefore, the catheter C may serve the dual-role of mapping of the target tissue and PF ablating the targeted tissue.

In the ablation configuration, the two adjacent electrodes of same polarity, for example combined electrode (A1, A1′) as well as combined electrode (A2, A2′) would have an equivalent length that may be greater than the length of one of the individual electrodes. For example, the combined ablation electrode comprising the electrodes A1, A1′ would have an equivalent length that may comprise two times the specific mapping electrode length d1 plus the inter-mapping spacing dE2. The equivalent length may thus be defined as: 2×d1+dE2. In the example, of the catheter 1 the equivalent length may thus be 3.5 mm (i.e., 2×1.5 mm+0.5 mm=3.5 mm). No gaps would be left in the lesion profile, for example of the cardiac tissue, when applied voltages exceed 2500 V, preferably 3500 V in the exemplary dimension of catheter 1. This is because the inter-pair distance dE1 may be designed to match the applied PFA waveform so that electric fields are caused within the tissue that exceed the thresholds required for irreversible electroporation (IRE) (e.g., of cardiac cells).

Catheter 2 is similar to catheter 1 but shows an example where the inter-mapping spacing dE2 between mapping electrodes A1, A1′ (and A2, A2′) is increased to about 1 mm, similarly as in catheter 3. Such configuration may be beneficial for longer targeted regions. Applied voltages via the pulses may need to be somewhat higher than for catheter 1 because of the increased electrode spacing of the mapping electrodes. Similarly, no gaps would be left in the lesion profile when applied voltages exceed 3000 V (preferably 3500 V). This is because the inter-pair spacing dE1 may be designed to match the applied PFA waveform so that together they yield electric fields that exceed the thresholds required for irreversible electroporation IRE.

In comparison catheter 3 may also be used to cover longer target areas. For the dimensions of catheter 3 no gaps would be left in the lesion profile when applied voltages via the pulses exceed 2500 V (preferably 3500 V). This is because the inter-pair spacing dE1 may be designed to match the applied PFA waveform so that the electric fields caused in the tissue exceed the thresholds required for irreversible electroporation IRE.

Catheters 4 and 5 are similar to catheter 3 (i.e. comprise pure ablation electrodes), but have additional mapping electrodes M1, M2 placed in between active (PFA) ablation electrodes A1, A2 (wherein a few electrode examples are marked in FIG. 1). For example, they may comprise six ablation electrodes, which form three mutually exclusive pairs P1, P3, P5. However, also pairs P2 and P4 generate a field that is sufficient for ablation such that an essentially gap-free ablation zone along the main axis (z) of the catheters 4, 5 can be generated. In addition to the pairs of mapping electrodes distally and proximally to the ablation electrodes, two further pairs of mapping electrodes M1, M2 may be provided, such that a mapping electrode pair M1, M2 is located in between each mutually exclusive pair of ablation electrodes P1. P3. P5. The benefit of such configurations may be that they can offer mapping information at locations in between active ablation electrodes A1, A2, for example. Additionally, compared to catheters 1 and 2, the hardware of the generator and the catheter can be simplified as there may be no switching required to combine two mapping electrodes M1, M2 into one active PFA ablation electrode, as needed for catheter 1 and catheter 2. However, higher voltages may be required to bridge over active PFA ablation electrodes A1, A2 that have mapping electrodes M1, M2 in between.

For example, for catheter 4 the system may be programmed to apply voltages >3000 V to the ablation electrode pairs P1, P3, P5 and voltages >3500 V to the ablation electrode pairs P2 and P4.

In the example of FIG. 1, the catheter 4 has a specific ablation electrode length d2 of about 3 mm, a specific mapping electrode length of about 1 mm, an inter-mapping spacing dE2 of about 1 mm. The interpair distance dE1 for ablation electrode pairs P1, P2, P3 is about 3 mm, respectively. The interpair distance dE1 for P2 and P4 is about 9 mm, respectively.

In the example of FIG. 1, the catheter 5 has a specific ablation electrode length d2 of about 3 mm, a specific mapping electrode length of about 1 mm, an inter-mapping spacing dE2 of about 1 mm. The interpair distance dE1 for ablation electrode pairs P1, P2, P3 is about 3 mm, respectively. The interpair distance dE1 for P2 and P4 may be reduced compared to catheter 4, e.g. to about 5 mm, respectively. Because of the decreased inter-electrode spacing, for catheter 4 the system may be programmed to apply voltages >2500 V to electrode pairs P1, P3, P5 and voltages >3000 V to electrode pairs P2 and P4.

It should be noted that due to the high voltages required for an ablation it should be avoided to apply the ablation pulses between electrodes M1, M2 of a pair of mapping electrodes. This may lead to high electrical fields that may cause electrolysis (which may cause bubble formation) and/or arcing. Also, the ablation electrodes should be configured to sustain the high electrical fields caused by the PFA pulses with the herein described voltages such that they are not damaged when thy apply the (PFA) pulse to the tissue.

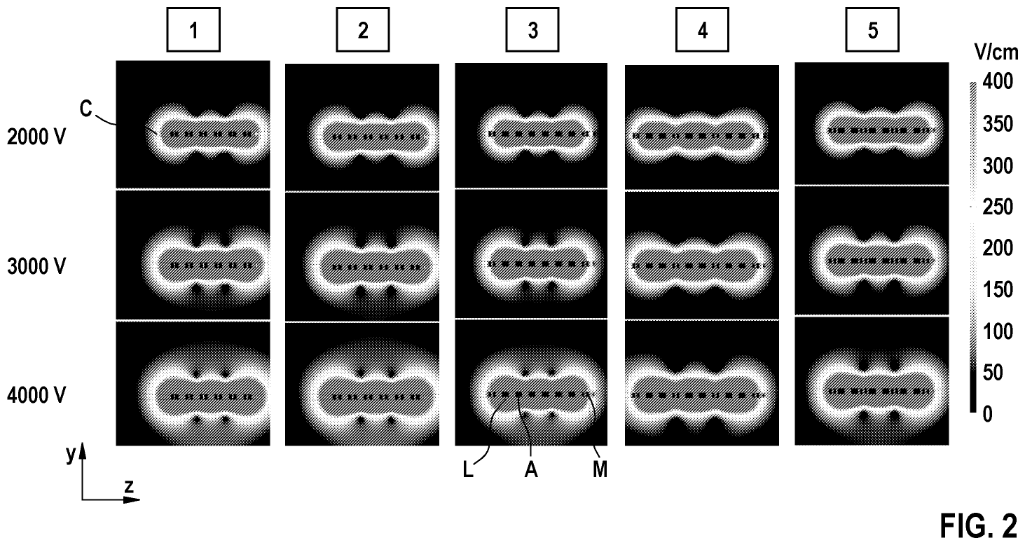

FIG. 2 shows simulation results for exemplary embodiments of the catheter representing the interaction of the exemplary catheters with the tissue for various pulse parameters. The simulation was based on a finite element analysis. Notably, FIG. 2 shows simulation results for the catheters 1-5 of FIG. 1. The column number represents the simulation results of the respective exemplary catheter (e.g., column 1 represents catheter 1, column 2 represents catheter 2, and so on). Each row represents the voltage applied to an ablation electrode pair in the simulation. The results are based on a static simulation of the electrical field, wherein the lower polarity (−) was set to ground, and the higher polarity (+) was set to the respective voltage, displayed for each row. In the static simulation the voltages applied to the ablation electrode pairs is applied simultaneously. The potential of the (non-marked) mapping electrodes, if they do not function as ablation electrodes, was set to a floating potential. The tissue surrounding the catheter in the simulation was chosen to represent the conductivity of a cardiac tissue (e.g., an atrial tissue).

For the simulation an ablation threshold of 400 V/cm was chosen since the ablation threshold of 400 V/cm may be assumed to be the IRE threshold for a cardiac tissue. The legend displaying the order of magnitude of the electrical field is thus limited to the ablation threshold. Hence, values above the ablation threshold are also displayed in the same tone as the ablation threshold of 400 V/cm. For medical and/or technical purposes it may suffice to display regions where the ablation threshold is exceeded since it indicates that these regions are ablated. The shape of the regions where the ablation threshold is exceeded can thus represent the shape of the lesion L generated by the catheter.

As can be seen in FIG. 2 catheters 1-5 with their ablation electrodes A and mapping electrodes M may all form lesions L of an elongated shape regardless of the indicated applied voltages (from 2000 V to 4000 V). Moreover, no gap formation within the lesion L is present such that an electrical isolation of the tissue can be ensured. With an increase of the applied voltages at the ablation electrodes the length of the lesion L in z-direction (e.g. along its main axis) may be slightly increased. Further, the voltage increase may slightly increase the lateral extent of the lesion L in the y-direction. As can be seen in the simulation results the lesion profile may comprise various dips in the lateral extent such that a slightly curved or undulating but elongated lesion profile may be generated in the tissue.

A catheter according to the invention may thus form an elongated lesion L with a length of at least 20 mm, at least 30 mm, at least 40 mm, at least 50 mm, at least 60 mm, at least 70 mm and/or at least 80 mm.

In an example, the lesion length may be increased by adding more pairs of ablation electrodes to the main axis of the catheter. For example, the catheter may also comprise at least 8, at least 10, at least 20, at least 30 pairs of ablation electrodes.

A catheter according to the invention may form a lesion with a maximum lesion width of at least below 7 mm, at least below 8 mm, at least below 9 mm, at least below 10 mm, at least below 12 mm and/or at least below 14 mm. However, the maximum lesion width may also be at least below 16 mm, at least below 18 mm and/or at least below 20 mm.

In another example, a catheter according to the invention may form a lesion L with a (maximum) length of at least 2 times the maximum lesion width (e.g. measured perpendicularly to its length), at least 3 times the maximum lesion width, at least 4 times the maximum lesion width, at least 5 times the maximum lesion width, at least 7 times the maximum lesion width and/or at least 10 times the maximum lesion width. The lesion may have an essentially linear shape.

The lesion L may be generated by the catheter on various tissues associated with the heart, for example, on the Crista Terminalis Isthmus CTI. In another example, the lesion L may be generated on an inner wall of the heart (e.g., an inner wall of an atrium, a ventricle, etc.). In a further example, the lesion L may be generated on an outer wall of the heart (e.g., an outer wall of the atrium, a ventricle, etc.). In summary, the lesion L may be generated in the endocardium, the myocardium and/or the epicardium of the heart. This may be enabled since the ablation catheter according to the invention may be formed as a single axis ablation catheter that may be easily positioned within the heart, as well, as on the outer wall of the heart.

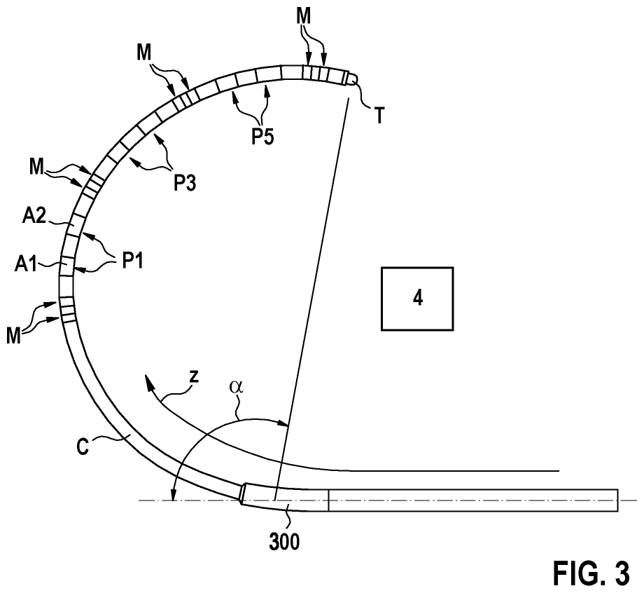

FIG. 3 shows an exemplary embodiment of a catheter C according to the invention with a curved main axis. Notably, the electrode configuration of catheter 4 is shown in FIG. 3. As can be seen, the portion of the main axis z that comprises the mapping electrodes M, and the pairs of ablation electrodes P1-P5 may be curled. As described herein, the main axis z may be steered to curl in a certain direction with respect to the initial point 300. The tangent of the initial point 300 of the catheter C, and a line connecting the initial point 300 with the tip T of the catheter, may span a curl angle α that may define the amount of the curl. In the example of FIG. 3 the curl angle α has a value of about 100°. However, the curl angle may be (limited) up to 5°, up to 10°, up to 45°, up to 90°, up to 135°, up to 180°. The curl of the main axis z may span a two-dimensional plane.

It may also be conceivable that a catheter C according to the invention may not be of a steerable construction. In that case a curl may, for example, not be present in the catheter C. In another example, the catheter may comprise a preset curl that may not be changed in contrast to the steerable example of the catheter C.

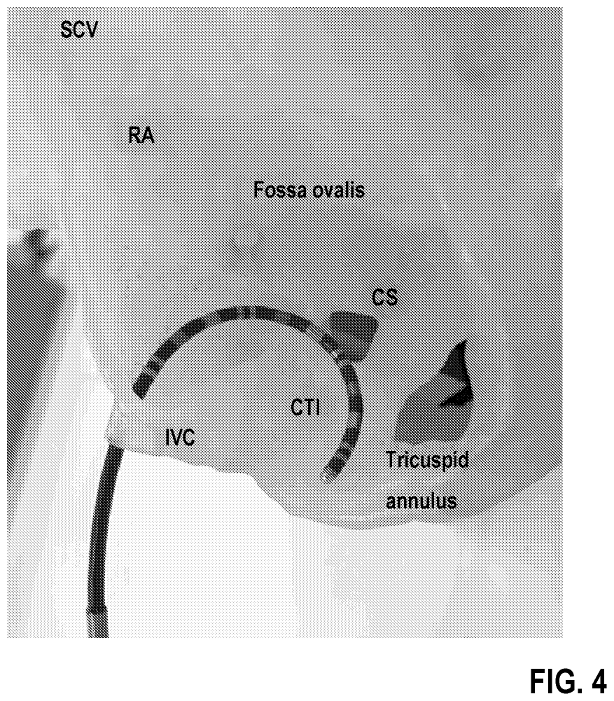

FIG. 4 shows a representation of an exemplary catheter ablation position in a heart model for an exemplary ablation treatment of the heart. In particular, a steerable catheter C according to the invention is shown that is deployed over the Crista Terminalis Isthmus CTI at the tricuspid valve annulus TVA. Furthermore, the heart model shows the positions of the superior vena cava SVC, the right atrium RA, the fossa ovalis, the coronary sinus CS, the inferior vena cava IVC. At such location, a one-shot PFA energy delivery according to the invention can achieve a complete cardiac conduction block.

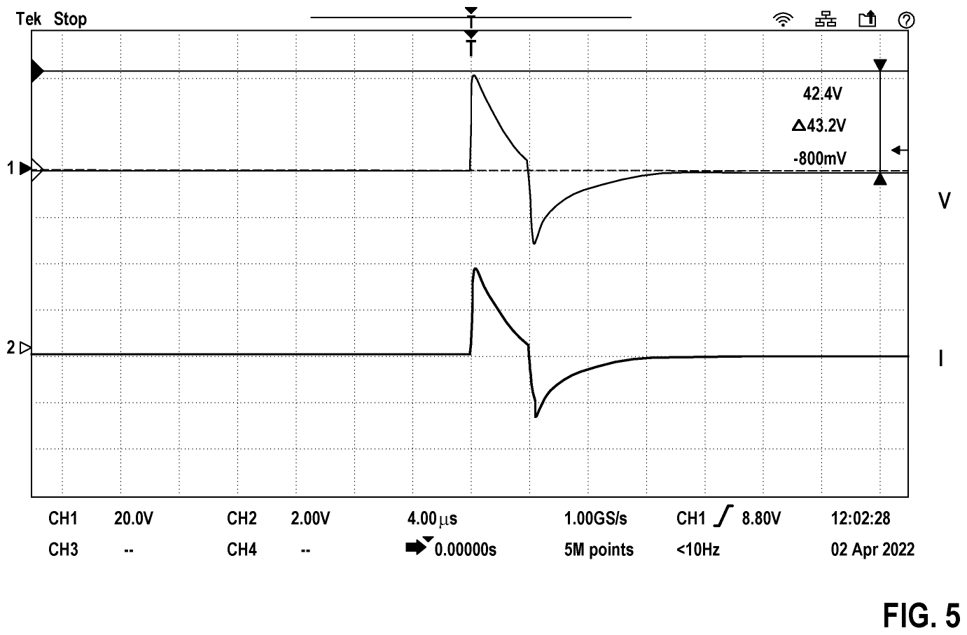

FIG. 5 shows a representation of an exemplary pulse which may be applied by a pair of ablation electrodes of the catheter for tissue ablation. In FIG. 5 channel 1 depicts the pulse voltage V which is scaled down by a factor of 100 (e.g., 40 V on channel 1 represent 4000 V of pulse voltage). The pulse may be biphasic, with a positive and a negative section. The amplitude pulse may peak at about +4300 V and at about −4000 V. Channel 2 depicts the resulting current I displayed as a voltage which is scaled down by a factor of 10 (e.g., 4 V on channel 2 represent 40 A of pulse current). The exemplary pulse shown on the oscilloscope display may represent a PFA waveform applied by the ablation electrodes with a peak at about +40 A and another peak at about −35 A. In some examples, the pulse amplitudes may be symmetric around zero.

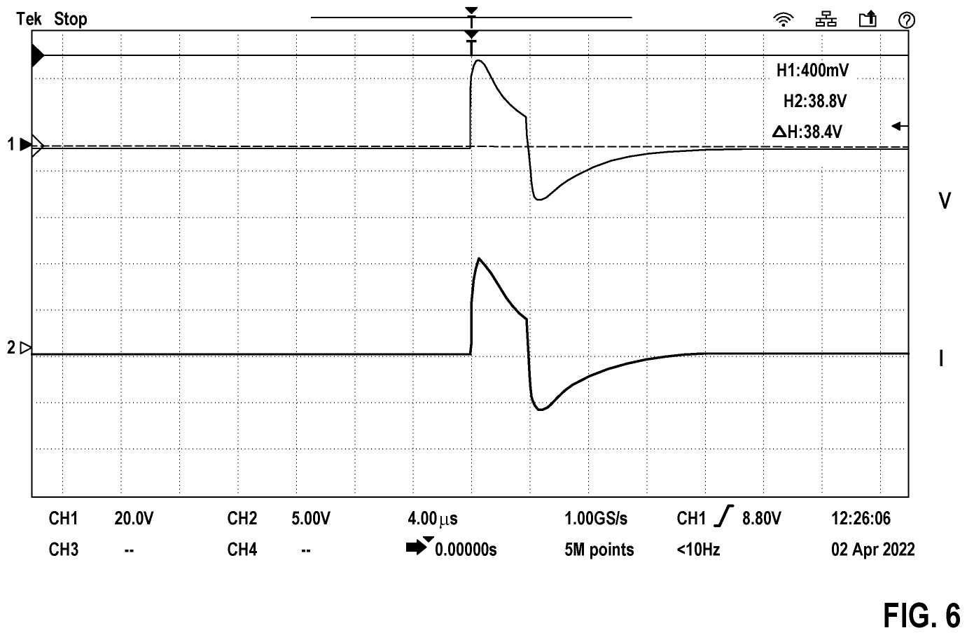

FIG. 6 shows a representation of another exemplary pulse which may be applied by a pair of ablation electrodes of the catheter for tissue ablation. The channel scaling of FIG. 6 corresponds to the channel scaling of FIG. 5. The exemplary pulse of FIG. 6 may thus represent another type of PFA waveform applied by the ablation electrodes. As can be determined from channel 1 (based on ΔH: 38.4 V) the voltage amplitude of the pulse is ±3840 V, i.e. slightly smaller than that outlined with reference to FIG. 5, but more symmetric around zero. Such a PFA waveform may thus be suitable for a pair of ablation electrodes that does not have mapping electrodes between them. In such a case, a lower voltage (compared to FIG. 5) may be sufficient since the mapping electrodes do not have to be bridged by the pulse.

Notably, a PFA waveform applied by the ablation electrodes may comprise a positive and a negative half-wave as can be seen in FIGS. 5 and 6. Each half wave may have a rectangular, sinusoidal and/or tooth shape. However, also other shapes may be conceivable. The maximum voltage of the PFA waveform may be between 1500 V and 5000 V. The pulse duration of the PFA waveform may be between 1 μs and 100 μs. The positive and negative half-waves may each comprise approximately half of the pulse duration. Instead of single pulses, also pulse trains with a certain pulse spacing may be applied. The PFA waveforms may have an increased safety profile as they may spare collateral tissues such as nerves, coronary arteries epicardial fat, esophagus, etc. Such PFA waveforms may also produce lesser thermal effects than other energy modalities (e.g. such as RFA treatments and/or cryogenic ablation treatments).

The delivered PFA waveforms may be matched to the electrode configuration of the catheter so that intended therapeutic effects are achieved without a need to reposition or move the catheter.

The PFA waveforms may be adjusted via a power unit that may be connected to the catheter. Via an according power electronic circuitry comprised in the power unit, a desired PFA waveform may be shaped and transmitted to the ablation electrodes.

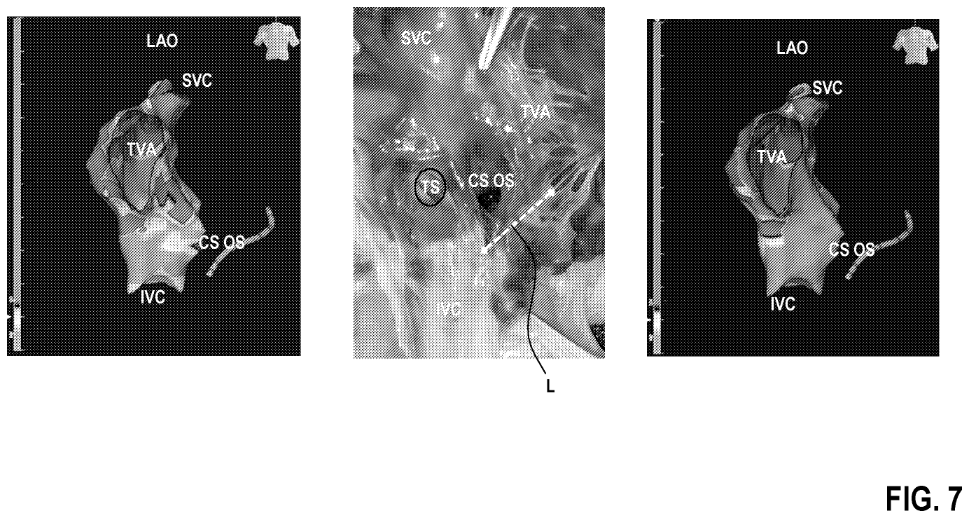

FIG. 7 is a representation of 3D voltage maps showing conductivity before (left) and after (right) an ablation procedure, as well as a pathology view of the ablation area. In the left image a pre-ablation 3D voltage map of a preclinical study is shown. The 3D voltage map shows a left anterior oblique LAO view of a heart section. The LAO view shows the superior vena cava SVC, the tricuspid valve annulus TVA, the inferior vena cava IVC, the coronary sinus ostium CS OS and a guiding catheter. For the ablation procedure the deployment of the catheter C was similar to the ablation position of the catheter shown in in FIG. 4.

In the right image of FIG. 7 a post-ablation 3D voltage map of the preclinical study is shown. As can be seen, the result of the ablation is a clear cardiac conduction block achieved at the CTI (at the right atrium) in one shot, without moving the catheter. The cardiac conduction block can be seen since the lower right region of the 3D map indicates no electrical activity taking place. The linear lesion L that may be ablated by the catheter C may thus, for example, be used to block a parasitic circular electrical activity at the CTI that can be seen in the left image.

For the ablation procedure, the PFA energy delivery took less than 30 μs to complete to achieve the shown conduction block. The block was also confirmed using the mapping electrodes of the catheter C.

The middle view of FIG. 7 shows a pathological overview of the one-shot lesion. The superior vena cava SVC, the tricuspid valve annulus TVA, the coronary sinus ostium CS OS, the inferior vena cava IVC are marked (as well as the Tricuspid Stenosis TS). Also, the position of the lesion L that was generated by the inventive catheter is shown.

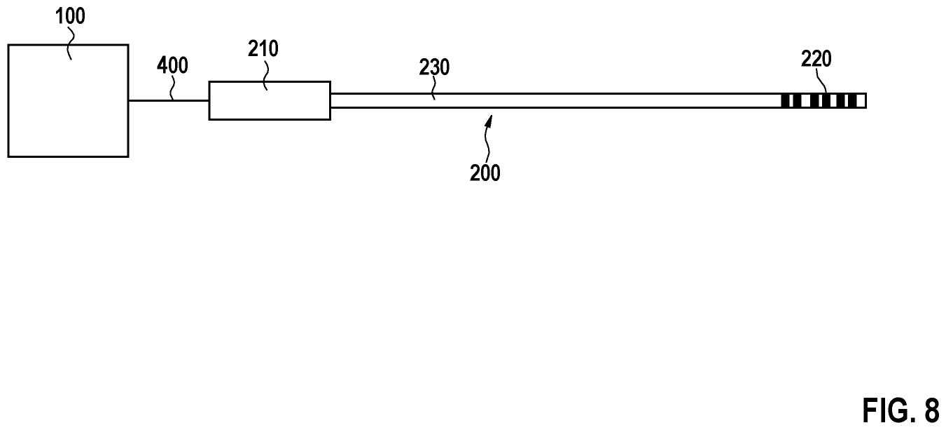

FIG. 8 shows a schematic overview of the whole system according to an embodiment of the invention. The generator 100 is configured for generating PF energy and the corresponding waveforms as described above. The generator 100 is connected via connector 300 to the catheter 200. The connector 400 could be configured as cable. The catheter comprises a proximal handle 210, a distal ablation portion 220 and a proximal catheter portion 230 between the handle 210 and the distal ablation portion 220. The distal ablation portion comprises 6 ablation electrodes arranged to three pairs.

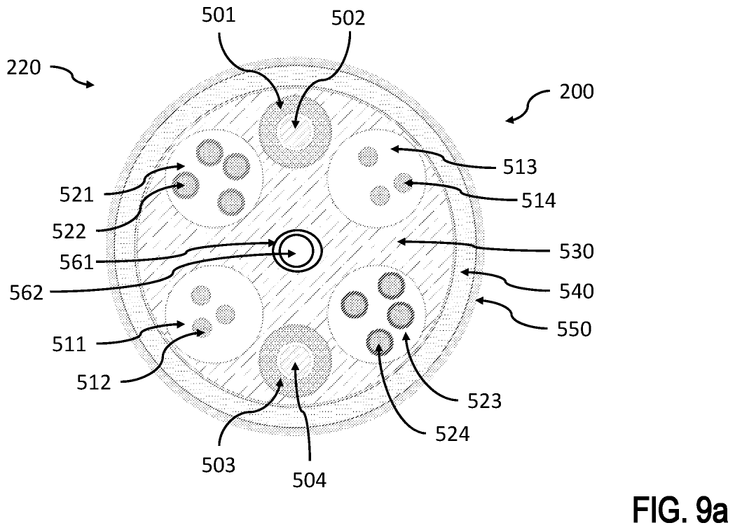

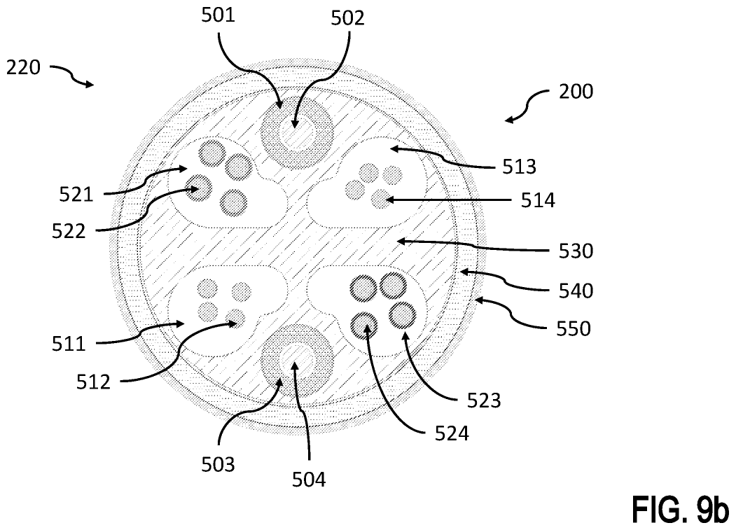

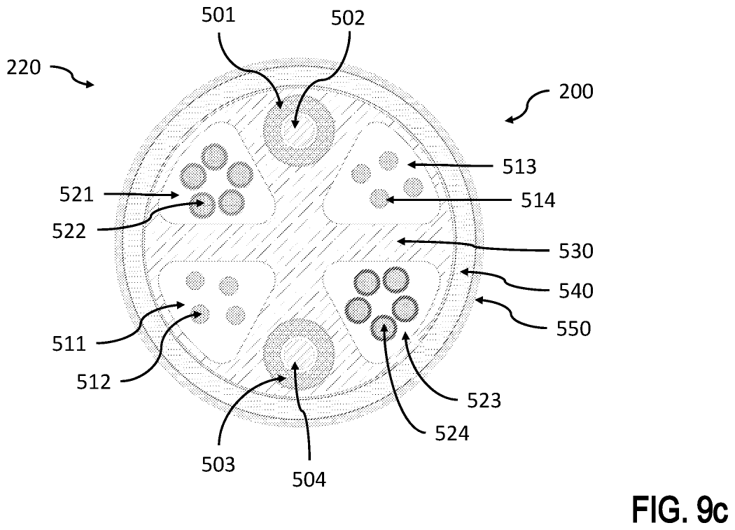

FIGS. 9a, 9b and 9c show schematic representations of exemplary embodiments of the lumen arrangement of a catheter according to the present invention. Each of the FIGS. 9a, b and c show a cross section of the distal ablation portion 220 of a catheter 200. Each exemplary catheter 200 comprises two lumens 501, 503 accommodating two pull wires 502, 504 used as steering elements (steering wires). One electrode 550 is shown in the cross section, whereby the electrode is arranged as a ring electrode on the catheter shaft 540. The electrode 550 is either a mapping electrode connected (not shown) to one of the mapping wires 512, 514 or an ablation electrode connected (not shown) to one of the ablation wires 522, 524. The mapping wires 512, 514 and ablation wires 522, 524 function as electrical conductors electrically connecting the respective mapping electrodes and ablation electrodes of the catheter 200 to the generator 100 via the connector 400 or to a mapping system (not shown). The space 530 between the individual lumen 501, 503, 511, 513, 521 and 523 is filled with an electrically isolating material, preferably the same material as the catheter shaft 540. Each ablation wire 522, 524 is covered with an electric insulator as well. Mapping wires 512 are in a separate lumen 511 than mapping wires 514 of opposing polarity. The same applies to ablation wires 522 and ablation wires 524 of the opposite polarity.

FIG. 9a shows a schematic representation of the cross section of the ablation portion 220 of a catheter 200 comprising three pairs of mapping electrodes and four pairs of ablation electrodes. Mapping wires 512, 514 and ablation wires 522, 524 of opposite polarity are arranged in separate lumens 511, 513, 521 and 523. Mapping wires 512, 514 do not share a lumen with ablation wires 522, 524. Furthermore, the ablation portion 220 of a catheter 200 comprises an additional central lumen 561 for a nitinol wire 562. The nitinol wire 562 is arranged in the ablation portion 220 to enhance the stiffness of this part of the catheter. Such an additional lumen 561 for a nitinol wire 562 could be used in all arrangement as shown in the FIGS. 9a-9c.

In the embodiment shown in FIG. 9a the lumens 511, 513, 521, 523 for the mapping wires and the ablation wires are arranged in a planetary like configuration, whereby each lumen 511, 513, 521, 523 is arranged at the same distance from the center of the cross section. This arrangement maximizes the distance between opposite polarities to withstand voltages in excess of 10 kV. Furthermore, it ensures a safe distance between the mapping wires and the ablation wires to avoid any pick-up of high-voltage from ablation wires by the mapping wires. Pick-up high voltages could cause field distortion, ineffective delivery of high voltage and/or shunting during the ablation procedure.

FIG. 9b shows an alternative arrangement of the lumens 511, 513, 521, 523 for the mapping wires and the ablation wires. In this embodiment the distance between opposite polarities is maximized as well. The mapping wires 514 and the ablation wires 522 share a polarity opposite to the polarity of the mapping wires 512 and the ablation wires 524. The X-like arrangement of the lumens 511, 513, 521, 523 improves the ability of the ablation section 220 to deflect in two opposite direction with respect to a plane crossing the center of the cross section and oriented in parallel to the straight sites of the lumens 511, 513, 521, 523.

FIG. 9c shows a pie-like arrangement of the lumens 511, 513, 521, 523 for the mapping wires and the ablation wires. In this arrangement mapping wires 512, 514 for four mapping electrodes are shown. Again, the mapping wires 514 and the ablation wires 522 share a polarity opposite to the polarity of the mapping wires 512 and the ablation wires 524.

In summary, the invention may enable treatment of cardiac conditions such as atrial flutter, persistent atrial fibrillation and/or ventricular tachycardia by ablating linear lesions via the herein described catheter of an according tissue. The system according to the invention may address the limitation of existing technologies by producing linear lesions without requiring catheter repositioning. The system according to the invention may comprise a catheter, a power unit (comprising a generator) and/or required accessories. The catheter may be positioned in place, at the targeted location, once and does not need to be repositioned until another targeted location is attempted. The accessories (e.g. cables, footswitch, a monitor, a keyboard, etc.) may connect the catheter to the generator, and/or may facilitate using the system by an operator. The generator of the power unit may deliver a PFA waveform with peak voltages sufficiently high to achieve required IRE thresholds for the given electrode configurations and geometries. The matching of waveform to catheter configurations may secure an effective therapy delivery without requiring catheter repositioning. In most case, the system according to this invention may achieve a desired therapy outcome by delivering just one PFA energy delivery shot. The mapping electrodes of the catheter may then be used to confirm the therapeutic result.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. Catheter (C, 200) for ablating a tissue using pulsed-field (PF) energy configured for connection to a high-voltage generator for generating PF energy, whereby the catheter comprises:
at least two ablation electrodes (A1, A2), in particular at least six ablation electrodes, configured for applying PF energy to the tissue;
wherein the catheter is configured such that, in an ablation position of the catheter, the ablation electrodes (A1, A2) contact the tissue along a main axis (z) of the catheter,
wherein the catheter comprises at least two separate electrical conductors to connect to said electrodes to said high-voltage pulse generator for generating PF energy,
wherein the at least two separate electrical conductors are adapted to deliver a waveform (W) with a peak voltage of at least 1000 V, in particular at least 3000 V, to said electrodes, and wherein the ablation electrodes of said catheter are spaced apart such that the electric field inside cardiac tissue exceeds an intensity of 400 V/cm at a depth of 5 mm when they are energized by said waveform.

2. Catheter according to claim 1, wherein the catheter is configured such that the tissue is ablated along a portion of the main axis, wherein the portion spans at least over a distance (dE1) between the ablation electrodes.

3. Catheter according to claim 1 or 2, wherein the catheter is configured such that an elongated profile (L) is ablated into the tissue.

4. Catheter according to any of claims 1-3, wherein at least one of the ablation electrodes is positioned at a sidewall of the catheter such that it is distanced to a tip of the catheter.

5. Catheter according to any of claims 1-4, wherein the catheter comprises two or more pairs of ablation electrodes (P1, P2, P3, P4, P5) positioned along the main axis (z).

6. Catheter according to claim 5, wherein the catheter is configured such that at least two of the pairs (P1, P2, P3, P4, P5) apply a pulse of an electrical energy substantially simultaneously to the tissue and/or such that at least two of the pairs apply separate pulses of the electrical energy to the tissue in the ablation position.

7. Catheter according to claim 5 or 6, wherein the two or more pairs of ablation electrodes are positioned along the main axis such that the application of the electrical energy in the ablation position causes a contiguous elongated profile (L) without gaps ablated in the tissue.

8. Catheter according to any of claims 1-7, wherein the catheter comprises one or more mapping electrodes (M1, M2) for sensing of the tissue.

9. Catheter according to claim 8, wherein the catheter is configured for switching a mapping electrode to function as an ablation electrode; and/or
switching an ablation electrode to function as a mapping electrode.

10. Catheter according to claim 9, wherein at least two adjacent mapping electrodes are arranged as pair of mapping electrodes having an inter-mapping spacing of less than 3 mm to function as an ablation electrode.

11. Catheter according to claim 10, wherein at two pairs of mapping electrodes are arranged on the catheter to function as a pair of ablation electrodes, whereby a first pair of adjacent mapping electrodes is arranged at an inter-pair distance (dE1) from the second pair of adjacent mapping electrodes.

12. Catheter according to claim 11, wherein six pairs of mapping electrodes arranged on the catheter to function as three pairs of ablation electrodes.

13. Catheter according to any of claims 8 to 12, wherein the catheter comprises at least one pair of mapping electrodes and one pair of ablation electrodes, wherein the electrical conductors connected to the mapping electrodes are in a separate lumen of the catheter than the electrical conductors connected to the ablation electrodes and wherein conductors connected to mapping and/or ablation electrodes of opposite polarity do not share a lumen.

14. Catheter according to any of claims 8 to 13, wherein two adjacent mapping electrodes are separated by an inter-mapping spacing is between 0.2 mm and 2.5 mm, in particular between 0.4 mm and 2.0 mm, in particular between 0.5 mm and 1.0 mm.

15. Catheter according to any of claims 8 to 14, wherein the mapping electrodes having a mapping electrode length (d1) of less than 3 mm, in particular between 0.5 mm and 2.5 mm, in particular between 0.5 mm and 2 mm, in particular between 1.0 mm and 1.5 mm.

16. Catheter according to any of claims 1 to 15, wherein two adjacent ablation electrodes are separated by an inter-pair distance (dE1) is at least 3 mm, in particular between 3 mm and 10 mm, in particular between 3 mm and 6 mm, in particular between 3 mm and 4 mm.
